(19) United States
(12) Patent Application Publication
(10) Pub. No.: US 2018/0188722 A1
(43) Pub. Date: Jul. 5, 2018

(54) MYOELECTRIC CONTROL OF UNMANNED AERIAL VEHICLE BY PROSTHETIC LIMB

(71) Applicant: International Business Machines Corporation, Armonk, NY (US)

(72) Inventors: Thomas D. Erickson, Minneapolis, MN (US); Kala K. Fleming, Nairobi (KE); Clifford A. Pickover, Westchester, NY (US); Komminist Weldemariam, Nairobi (KE)

(21) Appl. No.: 15/399,109

(22) Filed: Jan. 5, 2017

(51) Int. Cl. G05D 1/00; B64C 39/02

(52) U.S. Cl. CPC G05D 1/0016; A61F 2/54; B64C 39/024; G05D 1/0027

(57) ABSTRACT

A system for controlling an unmanned aerial vehicle (UAV). The system includes a prosthetic limb configured to receive myoelectric control signals from a user. The unmanned aerial vehicle is configured to perform an action responsive to the myoelectric control signals received by the prosthetic limb. For example, the action responsive to the myoelectric control signals may include flying to retrieve an object, flying to activate a switch, and providing a temperature reading to the user.

### BACKGROUND

The present invention is directed toward unmanned vehicles, and, more particularly, to controlling an unmanned aerial vehicle by a prosthetic limb.

A prosthesis is an artificial device that replaces a missing body part. A myoelectric prosthesis uses electromyography signals from muscle nerves within or close to a person's residual limb to control the movements of the prosthesis. The electromyography signals can be detected with electrodes located on the skin surface or implanted into tissue below the skin.

A myoelectric prosthesis typically simulates the user's missing limb. The prosthesis can have several joints actuated by electrical motors or servos, and the detected electromyography signals are used to control the electrical motors or servos. For example, a user's existing muscle contraction can signal the prosthetic elbow to bend, then use another contraction to signal the prosthetic hand to close.

In some cases, peripheral nerves of an amputated limb can be used to control the prosthetic limb. During a surgical procedure, nerve signals of the amputated limb are redirected to other muscles that can control the prosthesis. Then, when the user wants to move the prosthetic limb, the nerve signals originally used for limb movement are detected by electrodes and a control signal is sent to the prosthesis. Thus, a user can move the prosthesis just by choosing to move it.

### BRIEF SUMMARY

One example aspect of the present invention is a system that includes a prosthetic limb and an unmanned aerial vehicle. The prosthetic limb is configured to receive myoelectric control signals from a user. The unmanned aerial vehicle is configured to perform an action responsive to the myoelectric control signals received by the prosthetic limb.

Another example aspect of the present invention is a method for controlling an unmanned aerial vehicle. The method includes receiving myoelectric control signals from a user by a prosthetic limb. A performing operation performs an action by the unmanned aerial vehicle responsive to the myoelectric control signals received by the prosthetic limb.

Yet a further example aspect of the present invention is a computer program product for controlling an unmanned aerial vehicle. The computer program product includes computer readable program code configured to: receive myoelectric control signals from a user by a prosthetic limb and perform an action by the unmanned aerial vehicle responsive to the myoelectric control signals received by the prosthetic limb.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 shows an example system for controlling an unmanned aerial vehicle contemplated by the present invention.

FIG. 2 shows an electromyography system that may be used with the present invention.

FIG. 3 shows an example method for controlling an unmanned aerial vehicle, as contemplated by the present invention.

FIG. 4 shows an example computing environment used by embodiments of the present invention

### DETAILED DESCRIPTION

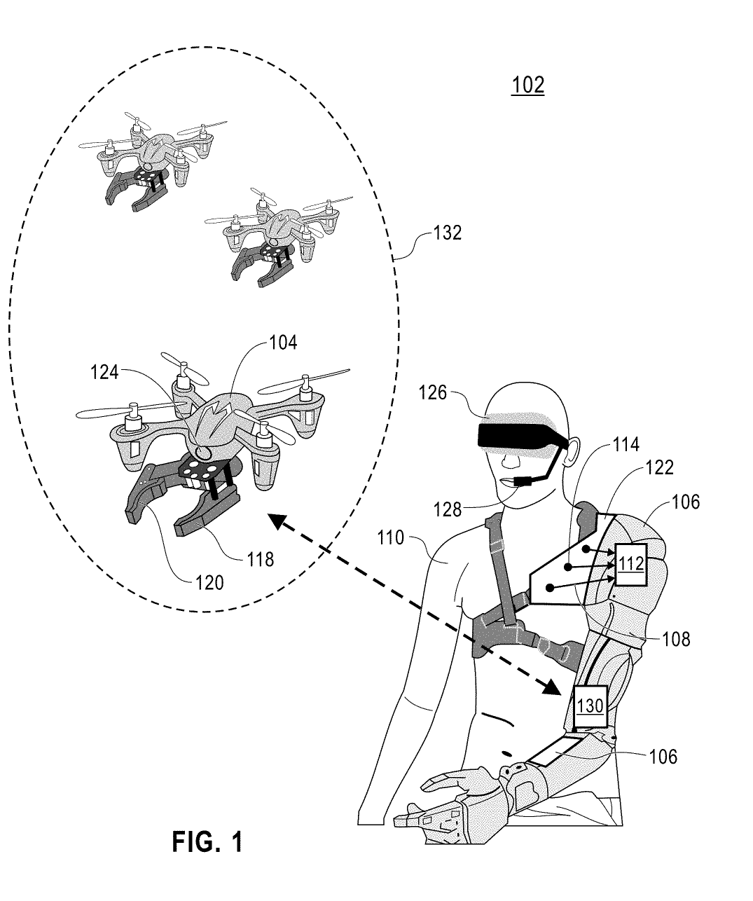

The present invention is described with reference to embodiments of the invention. Throughout the description of the invention reference is made to FIGS. 1-4. When referring to the figures, like structures and elements shown throughout are indicated with like reference numerals.

Aspects of the present invention include an unmanned aerial vehicle (UAV) (also referred to herein as a drone), a prosthetic limb responsive to myoelectric control signals from a user, and, based on the myoelectric control signals, the UAV performs an action. For example, a user can control a small drone on his or her prosthetic limb, which leaves the limb to turn off a light switch, provide video of something taking place outside the window of a home, provide a temperature reading from the kitchen, perform some fine physical task not easy to do with a prosthetic, or generally provide physical reality to the user.

FIG. 1 shows an example system 102 for controlling a UAV 104 contemplated by the present invention. The system 102 includes a prosthetic limb 106 configured to receive myoelectric control signals 108 from a user 110. In one embodiment, the prosthetic limb 106 includes a computer processor 112 that inputs and processes the myoelectric control signals 108. The UAV 104 is configured to perform an action responsive to the myoelectric control signals 108 received by the prosthetic limb 106.

In one embodiment, the system includes a plurality of targeted muscle reinnervation (TMR) electrodes 114 coupled to the prosthetic limb 106 and configured to detect electrical activity. The electrical activity is processed by the computer processor 112 and is used to control the UAV 104. TMR involves a surgical procedure where nerves in the residual limb are reattached to a healthy muscle elsewhere in the body, such as a chest muscle. The reattached nerves receive stimulation for the brain, but do not reach the amputated muscles. Instead, the TMR electrodes detect the nerves' electrical activity. This electrical activity is used to control the prosthetic limb 106 and/or the UAV 104. Thus, the user 110 can control the prosthetic limb 106 and the UAV 104 by choosing to move the amputated arm, wrist and hand.

In some embodiments of the present invention, the system 102 includes a myoelectric prosthesis control system with a gel liner that has a plurality of layers and a plurality of leads at least partially positioned between the plurality of layers as described in U.S. Pat. No. 9,155,634 issued Oct. 13, 2015 and incorporated herein by reference in its entirety. In addition, a plurality of electrodes can be coupled to the leads and portions of the electrodes can also be positioned between the plurality of layers. At least some of the electrodes can include an electrode pole that is configured to contact the residual limb to detect electromyographic signals.

As discussed above, the UAV 104 is configured to perform an action responsive to the myoelectric control signals 108. For example, the action responsive to the myoelectric control signals 108 may be flying to retrieve an object, flying to activate a switch, and/or providing a temperature reading to the user 110. Thus, the prosthetic limb 106 includes a transceiver 116 for transmitting control signals to the UAV 104. Various wireless communication protocols known to those skilled in the art, such as Bluetooth, Wi-Fi and ZigBee protocols, may be used by the system 102.

The transceiver 116 may also receive signals from the UAV 104. For example, the UAV 104 can include a gripper 118 with a tactile sensor 120. The tactile sensor 120 is used to convey a tactile signal to the user 110. A notification unit 122 is used to notify the user 110 of the tactile signal. For example, the notification unit 122 may signal the user 110 using tactile stimulation, such a vibrating motor. Other user notifications may be used by the notification unit 122, such as audio and visual signals that are proportional in intensity to the tactile signal intensity level from the UAV 104.

The UAV 104 can provide prosthetic hand haptic feedback and tactile feedback. For example, a three-dimensional force sensor may be installed on a region of the UAV 104. After the three-dimensional force sensor receives three-dimensional force input, the force sensor's output may be converted to a force signal transmitted back to the user 110 and/or the prosthetic limb 106. In one embodiment, the system 102 provides a pulse width modulated signal, via a haptic actuator drive circuit, such that the amplitude of the vibration of frequency provide tactile feedback to the user 110.

The UAV 104 may include a camera 124 that transmits a video stream. The system 102 may include a personal imaging system 126, such as smart glasses, worn by the user 110. Furthermore, the action responsive to the myoelectric control signals can include transmitting the video stream from the camera 124 carried by the UAV 104 to the personal imaging system 126.

The UAV 104 may have the ability to keep track, by storing locally or through a backend server, and learn many or all previous tasks or activities, and proactively remind the user 110. For instance, the user 110 may usually go twice a week to his or her garden to water flowers. The UAV 104 can learn this pattern of behavior from past several activities and remind the user 110 to water the flowers. This can be achieved, for example, through client-server communication between the UAV 104 and the backend services which the UAV 104 can retrieve via APIs.

In one embodiment, the system 102 may include a microphone 128 configured to receive voice commands from the user 110. The computer processor 112 can be configured to recognize the voice commands and control the UAV 104 in response to the voice commands. Thus, the user 110 may use voice commands, in addition to the myoelectric control signals 108, to control the UAV 104.

In one embodiment, the system 102 includes a docking port 130 on the prosthetic limb 106. The docking port 130 is configured to receive and secure the UAV 104 to the prosthetic limb 106. The docking port 130 may also be used as a charging station for the UAV 104.

The system 102 may include plurality of UAVs 104 coordinated to move in formation as a unit, flock or swarm. The flock 132 is responsive to the myoelectric control signals 108 received by the prosthetic limb 106. For example, the user 110 can control a flock 132 of five small UAVs. An extension movement of the prosthetic limb 106 can control the location of the flock 132, and movements of the fingers can bring the UAVs 105 closer or farther apart. Such a control method could allow the user 110 to pick up objects larger than a single UAV could handle, or perform operations involving two simultaneous actions, such as filling a cup from a faucet or opening a mailbox top and retrieving envelopes. The UAVs 104 may coordinate as cellular automata, initially taking direction from the user 110 but then coordinating among themselves given signals from the nearest neighbor.

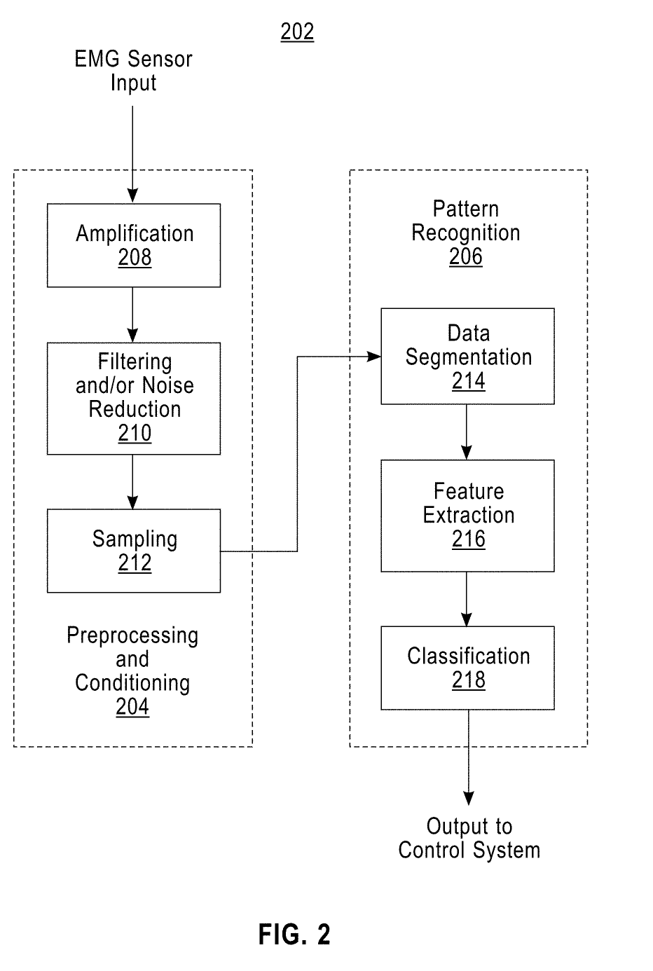

FIG. 2 shows an electromyography (EMG) system 202 that may be used with the present invention. The EMG system 202 includes a preprocessing and conditioning unit 204 and a pattern recognition unit 206. The preprocessing and conditioning unit 204 inputs an EMG signal from the user. The EMG signal may be provided by surface electrodes or from implantable myoelectric sensors (IMES).

The EMG signal from the user is amplified by an amplification unit 208. The amplified signal is passed to a filtering/noise reduction unit 210. The filtering unit/noise reduction 210 filters noise from the EMG signal. The filtered signal is then passed to the sampling unit 212. The sampling unit 212 captures digital samples of the EMG signal from processing in the pattern recognition unit 206.

The data segmentation unit 214 attempts to isolate or group meaningful parts of the EMG signal. Next, the feature extraction unit 216 uses pattern recognition and signal processing to determine meaningful feature vectors from the EMG signal. The feature vectors are then input to a classification unit 218. The classification unit 218 attempts to assign the feature vectors to one of a set of classes of actions to be performed in response to the EMG signal. The output of the classification unit 218 is then passed to a control system that carries out the function or functions classified by the classification unit 218.

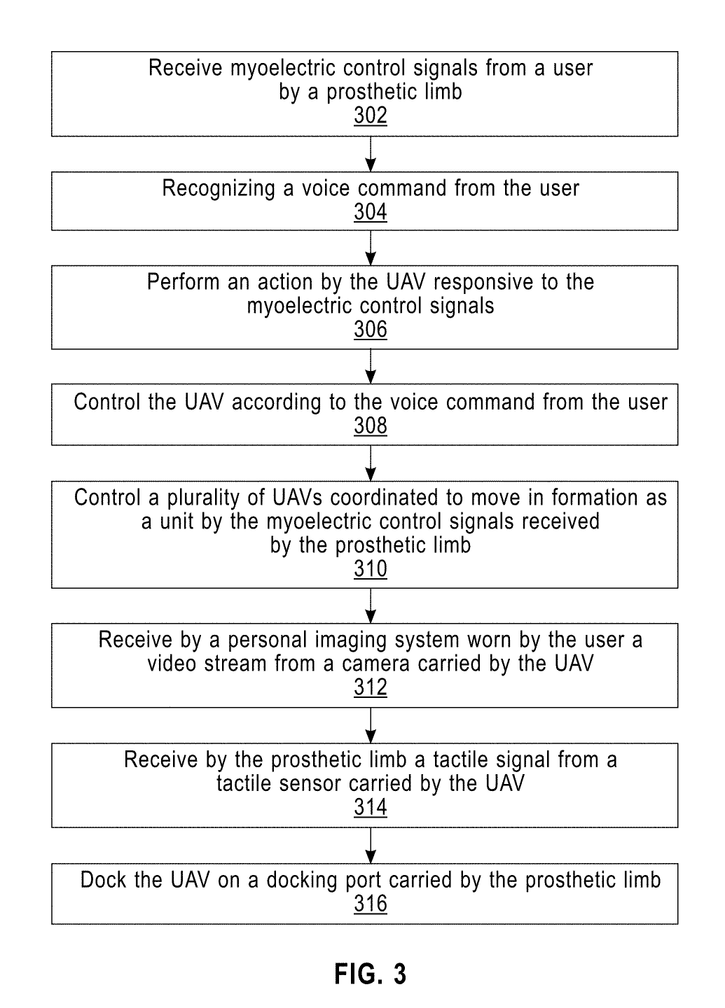

FIG. 3 shows an example method for controlling a UAV, as contemplated by the present invention. The method begins with receiving operation 302. During this operation, myoelectric control signals are received from a user by a prosthetic limb.

The prosthetic limb may be a TMR prosthesis, or may have implantable myoelectric sensors. A prosthetic arm includes myoelectric signal acquisition electrodes for acquiring, for example, finger unfolding and folding myoelectric signals. The myoelectric signals are input to a control module comprising a microprocessor and a motor driving circuit. The circuit controls unfolding and folding signals of the prosthetic hand, with the motor driving circuit driving a miniature direct-current motor to forward rotate or reverse rotate. A gear transmission mechanism drives the prosthetic hand to be folded or unfolded so as to drive the hand of the user to be unfolded and folded. As discussed below, the myoelectric signals are also used to deploy and/or control a UAV, with or without speech recognition to help direct the UAV.

The method 302 may include recognizing operation 304. During this operation, voice commands from the user are recognized. This operation requires a microphone and speech recognition software. Various methods for speech recognition known to those skilled in the art, including, Hidden Markov Models, dynamic time warping, and neural networks, may be used in embodiments of the present invention. Next, the method proceeds to performing operation 306.

At performing operation 306, the UAV performs an action responsive to the myoelectric control signals received by the prosthetic. The UAV action may include, for example, flying to retrieve an object, flying to turn off a light switch, flying to push a button, or to select and retrieve one object from a collection of objects. Such a system can give a user the ability to experience physical realities (e.g., to cultivate flowers, vegetation, farms, milking, driving, etc.) as opposed to virtual realities. Next, the method proceeds to controlling operation 308.

At controlling operation 308, the UAV is controlled according to the voice commands from the user. As discussed above, speech recognition may be used in combination with myoelectric signals to guide the UAV. The UAV may receive command (via speech or sound)) from the user to perform safety actions, such as when the user encounters a dangerous animal (e.g., snake) while in his or her garden or farm. Next, the method proceeds to controlling operation 310.

At controlling operation 310, a plurality of UAVs coordinated to move in formation as a unit are controlled by the myoelectric control signals received by the prosthetic limb. For example, a myoelectric signal meant to pinch the fingers together may cause a compaction of the spatial extent of a UAV swarm. In some cases, a person without a physical disability or challenge may use myoelectric signals to control a drone or drone swarm. Next, the method proceeds to receiving operation 312.

At receiving operation 312, a personal imaging system worn by the user receives a video stream from a camera carried by the UAV. The camera, mounted on the UAV, may be used to located or inspect a remote object, or see who is at the door. Such a camera-equipped UAV can be configured to use deep learning to learn to identify regularly needed objects such as pill bottles, tea cups or cell phones. After receiving operation 312, the method proceeds to receiving operation 314.

At receiving operation 314, the prosthetic limb receives a tactile signal from a tactile sensor carried by the UAV. Thus, the UAV can convey a tactile signal back to the user for a feeling of touch. It is contemplated that the UAV can provide other forms of feedback, such as speech, sound, blinking lights, etc. to convey information not suited to tactile feedback (e.g., proximity and temperature). If the UAV is equipped with a gripper, in one embodiment, the gripper provides tactile stimulus reception for use with the myoelectric prosthesis. The pressure transducer senses the level of pressure experienced by the gripping portion of the drone and converts the sensed pressure into a corresponding signal. A pressure stimulus member is positionable upon the user and creates a pressure stimulus proportionally corresponding to the sensed pressure of the pressure transducer by utilizing the signal from the pressure transducer. The pressure stimulus can be tactile so that the user can be given direct tactile pressure stimulation corresponding directly with the pressure sensed by the pressure transducer. After receiving operation 314, the method proceeds to docking operation 316.

At docking operation 316, the UAV is docked at a docking port carried by the prosthetic limb. The docking port may be used to both securely transport the UAV with the user and as a charging station for the UAV.

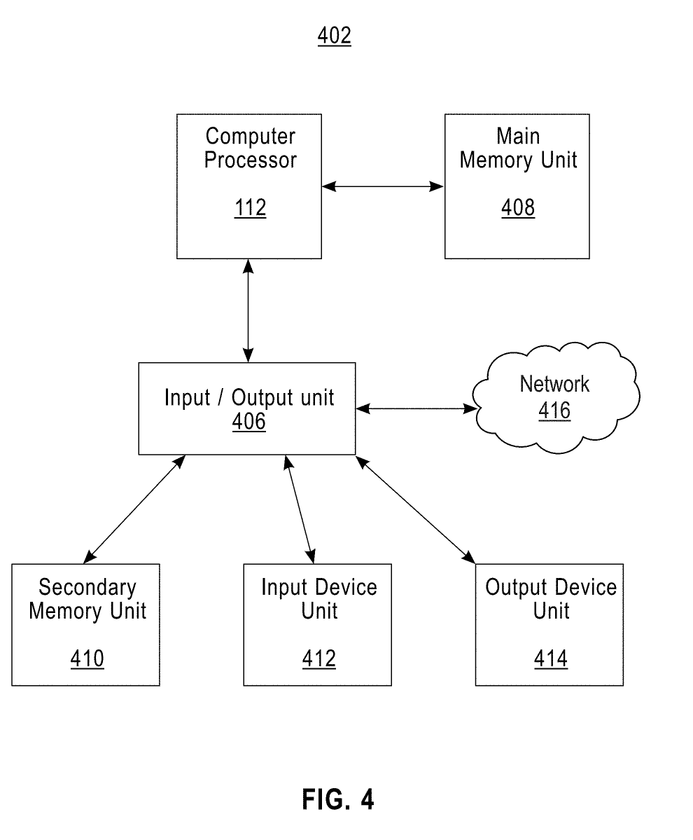

FIG. 4 shows an example computing environment 402 used by embodiments of the present invention. The computing environment 402 includes a computer processor 112 coupled to an input/output (I/O) unit 406, and a main memory unit 408. The main memory unit 408 generally stores program instructions and data used by the processor 112. Instructions and instruction sequences implementing the present invention may, for example, be embodied in the main memory unit 408. Various types of memory technologies may be utilized in the main memory unit 408, such as Random Access Memory (RAM), Read Only Memory (ROM), and Flash memory.

The I/O unit 604 connects with a secondary memory unit 410, an input device unit 412, and an output device unit 414. The secondary memory unit 410 represents one or more mass storage devices, such as hard disks, floppy disks, optical disks, and tape drives. Secondary memory 410 is typically slower than the main memory unit 408, but can store more information for the same price. The input device unit 412 may include input hardware such as a keyboard or mouse. The output device unit 414 typically includes devices such as a display adapter, a monitor and a printer. The I/O unit 406 may further be connected to a computer network 416.

Arrows in FIG. 4 represent the system bus architecture of the computer, however, these arrows are for illustrative purposes only. It is contemplated that other interconnection schemes serving to link the system components may be used in the present invention. For example, a local video bus could be utilized to connect the computer processor 112 to an output device 414, even though a direct arrow between the computer processor 112 and the output device 414 is not shown.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

As will be appreciated by one skilled in the art, aspects of the present invention may be embodied as a system, method or computer program product. Accordingly, the present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A system comprising:
a prosthetic limb configured to receive myoelectric control signals from a user; and
an unmanned aerial vehicle (UAV) configured to perform an action responsive to the myoelectric control signals received by the prosthetic limb.

2. The system of claim 1, further comprising a docking port carried by the prosthetic limb, the docking port configured to receive the UAV.

3. The system of claim 1, wherein the action responsive to the myoelectric control signals includes at least one of flying to retrieve an object, flying to activate a switch, and providing a temperature reading to the user.

4. The system of claim 1, further comprising:
a personal imaging system configured to be worn by the user; and
wherein the action responsive to the myoelectric control signals includes transmitting a video stream from a camera carried by the UAV to the personal imaging system.

5. The system of claim 1, further comprising:
a tactile sensor carried by the UAV for conveying a tactile signal to the user; and
a notification unit configured to notify the user of the tactile signal.

6. The system of claim 1, further comprising:
a microphone configured to receive voice commands from the user;
a computer processor coupled to the microphone and configured to recognize the voice commands and control the UAV in response to the voice commands.

7. The system of claim 1, further comprising a plurality of targeted muscle reinnervation (TMR) electrodes coupled to the prosthetic limb and configured to detect electrical activity, the electrical activity used to control the UAV.

8. The system of claim 1, further comprising a plurality of UAVs coordinated to move in formation as a unit, the unit responsive to the myoelectric control signals received by the prosthetic limb.

9. A method for controlling an unmanned aerial vehicle (UAV), the method comprising:
receiving myoelectric control signals from a user by a prosthetic limb; and
performing an action by the UAV responsive to the myoelectric control signals received by the prosthetic limb.

10. The method of claim 9, further comprising docking the UAV on a docking port carried by the prosthetic limb.

11. The method of claim 9, wherein the action responsive to the myoelectric control signals includes at least one of flying to retrieve an object, flying to activate a switch, and providing a temperature reading to the user.

12. The method of claim 9, further comprising receiving by a personal imaging system worn by the user a video stream from a camera carried by the UAV.

13. The method of claim 9, further comprising receiving by the prosthetic limb a tactile signal from a tactile sensor carried by the UAV.

14. The method of claim 9, further comprising:
recognizing a voice command from the user; and
controlling the UAV according to the voice command from the user.

15. The method of claim 9, further comprising controlling a plurality of UAVs coordinated to move in formation as a unit by the myoelectric control signals received by the prosthetic limb.

16. A computer program product for controlling an unmanned aerial vehicle (UAV), the computer program product comprising:
a non-transitory computer readable storage medium having computer readable program code embodied therewith, the computer readable program code configured to:
receive myoelectric control signals from a user by a prosthetic limb; and
perform an action by the UAV responsive to the myoelectric control signals received by the prosthetic limb.

17. The computer program product of claim 16, further comprising computer readable program code configured to:
recognize a voice command from the user; and
control the UAV according to the voice command.

18. The computer program product of claim 16, further comprising computer readable program code configured to control a plurality of UAVs coordinated to move in formation as a unit by the myoelectric control signals received by the prosthetic limb.

19. The computer program product of claim 16, further comprising computer readable program code configured to receive by the prosthetic limb a tactile signal from a tactile sensor carried by the UAV.

20. The computer program product of claim 16, wherein the action responsive to the myoelectric control signals includes at least one of flying to retrieve an object, fly to activate a switch, and providing a temperature reading to the user.
